(19) United States
(12) Patent Application Publication
(10) Pub. No.: US 2013/0228179 A1
(43) Pub. Date: Sep. 5, 2013

(54) AIRWAY MANAGEMENT APPARATUS

(71) Applicant: Frank J. Fischer, JR., Bloomington, IN (US)

(72) Inventor: Frank J. Fischer, JR., Bloomington, IN (US)

(73) Assignee: Cook Medical Technologies LLC, Bloomington, IN (US)

(21) Appl. No.: 13/412,042

(22) Filed: Mar. 5, 2012

(51) Int. Cl. A61M 16/04; A61M 16/00; A61M 16/20

(52) U.S. Cl. 128/204.18; 128/207.14; 128/207.16

(57) ABSTRACT

An airway management apparatus for engagement with a catheter for oxygenation of a patient includes a main body having a major axis, a first port extending in a proximal direction along the major axis, a second port, and a third port extending in a distal direction along the major axis. The third port comprises a connector for engagement with a proximal end of the catheter. The connector may have a plurality of radially compressible members extending in a distal direction, and circumferentially aligned to receive the catheter proximal end. An axially movable member is positioned to selectively compress the compressible members around the catheter proximal end to form a locking engagement, and to release the compressible members. A wire guide is insertable into the trachea through the first and third ports, and the catheter.

### BACKGROUND OF THE INVENTION

1. Technical Field

The present disclosure relates generally to airway management devices. More particularly, the disclosure relates to an airway management apparatus for use in oxygenating a patient during endotracheal tube intubation and/or extubation.

2. Background Information

Airway exchange catheters are often used to oxygenate a patient during endotracheal tube (ETT) exchange. Removal of an endotracheal tube from the trachea of a patient is commonly referred to as extubation. Insertion of an endotracheal tube is commonly referred to as intubation. After an ETT has been positioned in the trachea of the patient for a period of time, a physician may determine that the existing ETT should be removed and exchanged for a new ETT, or in some instances, cleaned and repositioned in the trachea. The necessity to remove an existing ETT from the trachea of a patient and replace it with a new, or a cleaned, ETT may arise from, among other things, the physician's desire to utilize an ETT of a different size, the displacement of the existing ETT, or the malfunction of the existing ETT resulting from conditions such as blockage, e.g., as may be caused by patient mucous.

Proper placement and use of airway exchange catheters during endotracheal tube replacement is well known in the art. One particularly well-known method for replacing an ETT while maintaining oxygenation of the patient via an airway exchange catheter is described in U.S. Pat. No. 5,052,386, incorporated by reference herein. According to the method described in the '386 patent, the existing ETT is disconnected from a ventilator, and the airway exchange catheter is connected to the ventilator by way of a removable connector at the proximal end of the airway exchange catheter. The catheter is then inserted into the lumen of the placed endotracheal tube. The connector is configured to allow rapid connection, and disconnection, between the airway exchange catheter and the ventilator. The airway exchange catheter may be disconnected from the ventilator via the removable connector as the ETT is removed from about the catheter. A replacement ETT may then be inserted over the airway exchange catheter, and the catheter is reconnected to the ventilator utilizing the removable connector. Once the replacement ETT is determined to be properly positioned in the trachea, the airway exchange catheter is disconnected from the ventilator and removed from the interior space of the ETT. The ventilator is then connected to the replacement ETT.

It is sometimes desirable to position a wire guide in either the left or right mainstem bronchus during airway management, and to maintain the wire guide in this position during the course of treatment. Maintaining a wire guide in this manner secures access to the desired airway bronchus, and thereby facilitates the later access of a working catheter (e.g., an endobronchial blocker catheter) by providing a conduit into the bronchus. When an airway exchange catheter is inserted into an airway which has previously been secured by a wire guide, the wire guide extends through the lumen of the airway exchange catheter. However, when it is desired to oxygenate the patient via the airway exchange catheter having the removable connector on the proximal end of the catheter as described above, the presence of the wire in the catheter lumen obstructs the passage of the ventilating fluid therethrough. Additionally, the presence of the wire extending out the proximal end of the airway exchange catheter obstructs the ability to securely connect the airway exchange catheter to the ventilator via the removable connector described above.

It is desired to provide an apparatus for use in airway management, such as endotracheal tube replacement, that overcomes the problems associated with prior art catheters. More particularly, it is desired to provide an apparatus that permits use of a removable connector with an airway exchange catheter that is suitable for oxygenating the patient even when the catheter has been inserted over a wire guide.

### BRIEF SUMMARY

The present invention addresses the shortcomings of the prior art. In one form thereof, the invention comprises an airway management apparatus for engagement with a catheter for oxygenation of a patient. The airway management apparatus includes a generally hollow main body having a plurality of ports open to an interior space thereof. The main body has a major axis, a first port extending in a proximal direction from the main body along the major axis, a second port angularly offset from the major axis, and a third port extending in a distal direction from the main body wherein the third port is at least substantially in-line with the first port along the major axis. The third port comprises a connector for engagement with a proximal end of the catheter. The connector comprises a plurality of radially compressible members extending in a distal direction, wherein the compressible members are circumferentially aligned to define a chamber for receiving the catheter proximal end. The third port further comprises an axially movable member positioned for selectively compressing a distal end portion of the compressible members around the catheter proximal end, and for releasing the compressible members from around the catheter proximal end.

In another form thereof, the invention comprises an airway management system for use in oxygenating a patient, such as during endotracheal tube cleaning and/or replacement. A catheter has a proximal end, a distal end, a passageway extending therethrough, and an outer surface dimensioned to be received in a passageway through an endotracheal tube. An airway management apparatus comprises a generally hollow main body and a plurality of ports open to an interior space of the main body. The plurality of ports includes a first port disposed at a proximal portion of the apparatus, a second port, and a third port disposed at a distal portion of the apparatus. The third port is positioned substantially in-line with the first port along an axis of the apparatus. The third port comprises a connector sized and configured for releasably engaging the proximal end of the catheter. A wire guide may be configured to extend through the first port and the third port along the axis, and through the catheter when the catheter is engaged with the third port.

In still another form thereof, the invention comprises a method for oxygenating a patient during removal of an endotracheal tube, wherein a proximal end of the endotracheal tube is engaged with a ventilation apparatus and a distal end extends into the trachea of the patient. A multi-port airway apparatus is positioned for engagement with a catheter. The multi-port airway apparatus comprises a main body having a proximal first port, a second port, and a distal third port. The third port is at least substantially in-line with the first port, and comprises a connector member. A proximal end of the catheter is engaged with the connector member of the third port. The proximal end of the endotracheal tube end is disengaged from the ventilation apparatus, and the ventilation apparatus is engaged with the second port. A distal end of the catheter is passed through a passageway of the endotracheal tube, such that the catheter distal end extends beyond the distal end of the endotracheal tube. A guide device is passed through the first and third ports of the multi-port airway apparatus, and through a lumen of the catheter, such that a distal end of the guide device extends into the trachea. The endotracheal tube may be at least partially withdrawn from the trachea over the catheter and guide device. The proximal end of the catheter may be disengaged from the connector member of the third port, the airway apparatus may be withdrawn over the guide device, and the endotracheal tube may be withdrawn over the guide device. The airway apparatus may be re-aligned over the guide device, and the catheter proximal end may be re-engaged with the connector member to re-establish a flow of ventilating fluid to the patient.

### BRIEF DESCRIPTION OF THE DRAWINGS

FIG. 1 is a side view of a prior art connector that has been connected to the proximal end of an airway exchange catheter;

FIG. 2 is a side view of the prior art connector of FIG. 1, wherein the collar has been retracted to release the catheter from the connector;

FIG. 3 is a side view of an airway management apparatus according to an embodiment of the present invention;

FIG. 4 is a side view of the airway management apparatus as shown in FIG. 3, showing an airway exchange catheter locked at the distal end of the apparatus and a wire guide extending through the apparatus;

FIG. 5 is a longitudinal sectional view of the airway management apparatus with a catheter in the locked position as shown in FIG. 4;

FIG. 5A is an enlarged view of a portion of the distal port comprising a plurality of axially-extending fingers;

FIG. 5B is an enlarged view of a portion of the distal port comprising an axially slidable collar;

FIG. 6 is a longitudinal sectional view sectional view of the airway management apparatus and catheter of FIG. 4 in an unlocked position;

FIG. 7 is an alternative embodiment of the airway management apparatus of FIG. 4, also showing an airway exchange catheter locked at the distal end of the apparatus and a wire guide extending through the apparatus; and

FIG. 8 illustrates use of the airway management apparatus during exchange of an endotracheal tube in a patient.

### DESCRIPTION OF THE PREFERRED EMBODIMENTS

For purposes of promoting an understanding of the present invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

In the following discussion, the terms “proximal” and “distal” will be used to describe the opposing axial ends of the airway management apparatus, as well as the axial ends of various components. The term “proximal” is used in its conventional sense to refer to the end of the apparatus (or component) that is closest to the operator during use of the apparatus. The term “distal” is used in its conventional sense to refer to the end of the apparatus (or component) that is initially inserted into the patient, or that is closest to the patient during use.

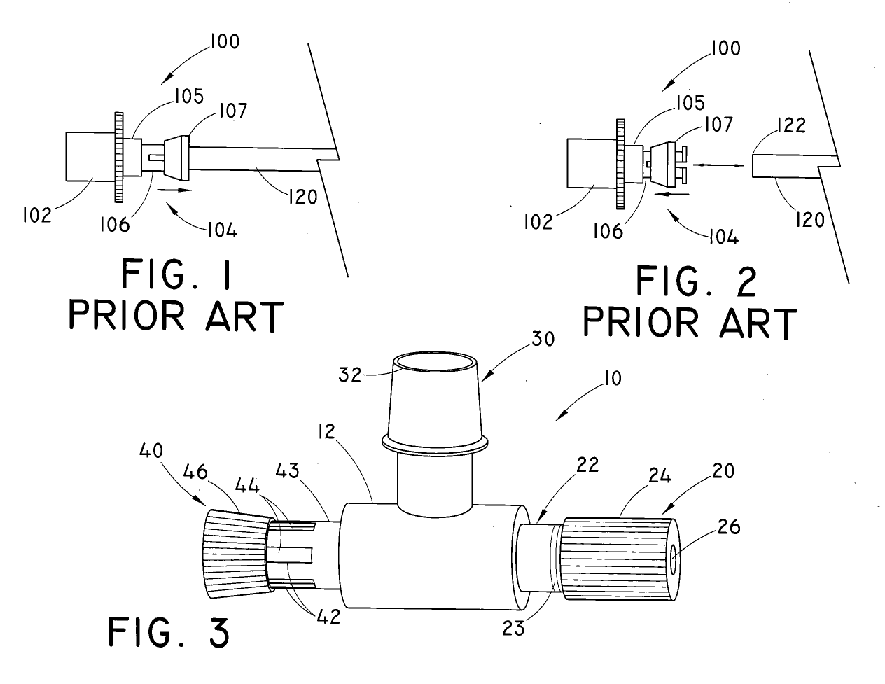

FIG. 1 is a side view of a prior art removable connector 100 of the type disclosed in the incorporated-by-reference U.S. Pat. No. 5,052,386. In FIG. 1, collar 107 has been advanced such that connector 100 is engaged with the proximal end of an airway exchange catheter 120. FIG. 2 is another side view of the prior art connector of FIG. 1. As explained below, collar 107 is retracted in FIG. 2 to release catheter 120 from the connector.

In the example shown herein, the prior art removable connector of FIGS. 1 and 2 includes a conventional 15 mm ventilator fitting portion 102 at a proximal end. Fitting portion 102 is sized and configured for connection to a mating fitting of a mechanical ventilation apparatus (not shown), in well-known fashion. A catheter fitting portion 104 is provided at the distal end of the connector for connection to the proximal end 122 of airway exchange catheter 120. Catheter fitting portion 104 comprises a sleeve 105 having a plurality of fingers 106 extending in an axial direction therefrom. Fingers 106 are aligned in the catheter fitting portion in a manner to comprise a chamber for snugly receiving catheter proximal end 122. Slidable collar 107 is positioned about fingers 106. Collar 107 is axially movable (in the direction of the arrows in respective FIGS. 1 and 2) between a distal (locked) position for engaging the catheter 120 as shown in FIG. 1, and a proximal (unlocked) position as shown in FIG. 2 for disconnecting the catheter from connector 100.

Some removable connectors known in the art include other known fittings in place of the 15 mm fitting portion 102. For example, a threaded Luer lock fitting may be used in place of the 15 mm fitting portion. Such Luer lock fittings are commonly used, e.g., for connection to a jet ventilator when jet ventilation is desired instead of conventional mechanical ventilation. Removable connector 100 is typically formed of a hard plastic material, such as polycarbonate. Connectors such as those described above are commercially sold by Cook Medical, of Bloomington, Ind., as RAPI-FIT® connectors. Further description of removable connector 100 is provided in the incorporated-by-reference '386 patent.

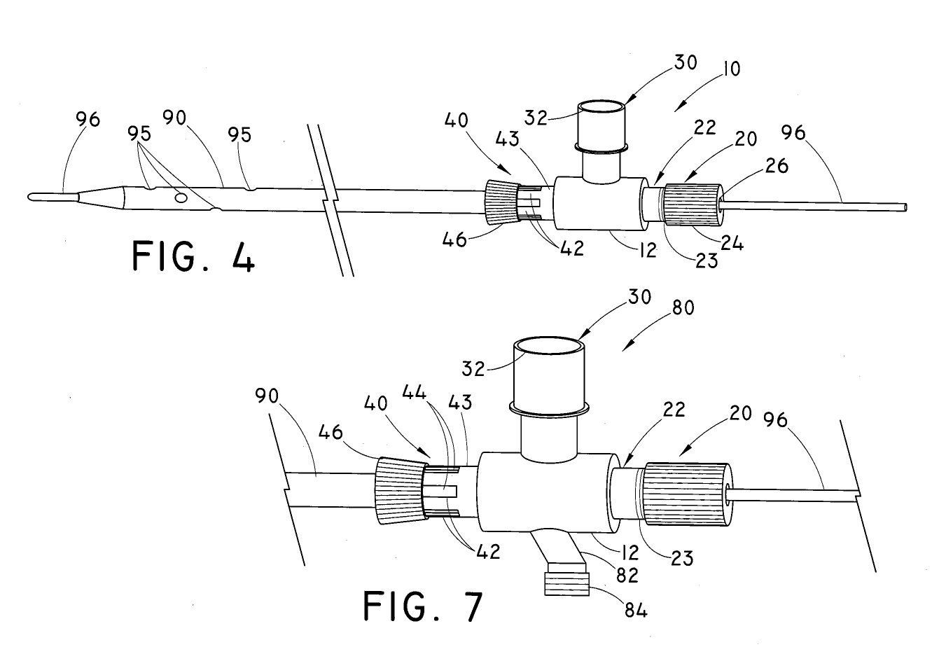

FIG. 3 is a side view of an airway management apparatus 10 according to an embodiment of the present invention. Airway management apparatus 10 includes a generally hollow main body 12, and a plurality of ports that open to the generally hollow interior of main body 12. In the non-limiting embodiment shown in FIG. 3, main body 12 includes ports 20, 30, and 40.

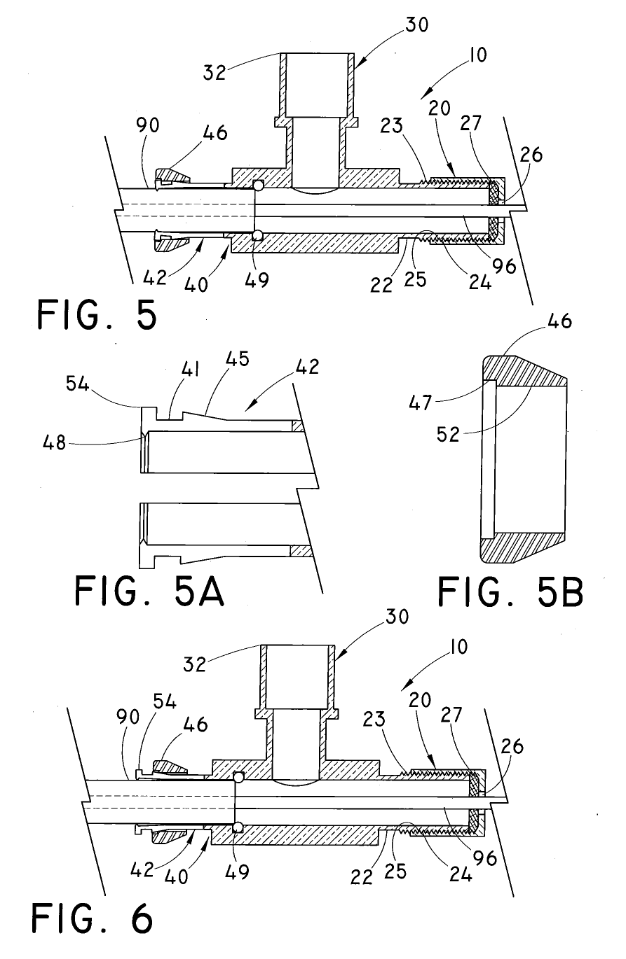

Port 20 extends in a proximal direction along main body 12. In this embodiment, port 20 comprises a tubular member 22. Preferably, at least a portion of the length of tubular member 22 is provided with external threads 23. An end cap 24 having internal threads 25 is received over the external threads of tubular member 22. External threads 23 and internal threads 25 are sized and aligned for threaded engagement in well-known fashion along mating surfaces, e.g., as shown in FIGS. 5 and 6. Those skilled in the art will appreciate that other known means of engaging the tubular member and end cap may be substituted. An opening 26 extends through end cap 24, and communicates with the hollow interior of main body 12. In a preferred embodiment, a valve member, such as a check-flow disk-type valve 27 (FIGS. 5, 6), a Tuohy valve, or other conventional valve, is provided internally of end cap 24 in well-known fashion to establish a fluid-tight connection with main body 12.

Port 30 extends at substantially a 90 degree angle from the major axis of main body 12. In the embodiment shown, port 30 is equipped with a conventional 15 mm connector 32 for connection to a conventional mechanical ventilator (not shown). Although port 30 preferably extends at an angle of about 90 degrees from the major axis as stated, those skilled in the art will appreciate that other angles between about 10 degrees and 170 degrees may be substituted, as long as the position of port 30 does not functionally interfere with ports 20 and 40, as described herein. Other conventional connectors, such as a Luer connector suitable for engagement with a mating connector of, e.g., a jet ventilator, may be substituted for the 15 mm ventilation connector 32 shown in the figures.

Port 40 is located opposite and functionally in-line with port 20 along the major axis of main body 12. Although it is preferred that ports 20 and 40 are axially in-line with each other along the major axis of apparatus 10, ports 20 and 40 need not necessarily be exactly axially in-line in all instances. However, it is preferred that they are at last substantially in-line (e.g., not axially offset by more than about 20 degrees). Port 40 is configured, e.g., as a connector for engaging the proximal end of a device, such as an airway exchange catheter 90. As shown in FIGS. 4-6 and as described herein, this connector may be constructed in a manner to rapidly connect, and disconnect, the airway exchange catheter 90 from airway management apparatus 10. In the non-limiting embodiment shown and described, port 40 is configured such that the proximal end of the airway exchange catheter is connected to the port in a manner similar to that shown in the incorporated-by-reference '386 patent. In this embodiment, port 40 comprises a radially compressible mechanism, such as the plurality of fingers 42 that extend in the axial direction from a generally cylindrical base portion 43. Fingers 42 are separated by slots 44, and are circumferentially aligned in manner to define a chamber for snugly receiving the proximal end of the catheter. An axially slidable collar 46 is positioned about fingers 42.

FIG. 4 illustrates the airway management apparatus 10 as shown in FIG. 3, engaged with an airway exchange catheter 90. Airway exchange catheter 90 preferably includes one or more side ports 95 along a distal end of the catheter. Side ports 95 permit the ventilation fluid (e.g., oxygen) to pass through the catheter to oxygenate the patient even when a wire guide 96 or like device extends through the catheter, as shown in the figure. Airway exchange catheters are well known in the art, and are described, for example, in the incorporated-by-reference '386 patent. Further description of the airway exchange catheter is not necessary for an understanding of the present invention, as those skilled in the art are capable of selecting a suitable airway exchange catheter or like device for use herein, when applying the teachings of the present invention.

A small diameter guide device, such as wire guide 96, extends through the apparatus. Although the small diameter apparatus is described and shown herein as a wire guide, those skilled in the art will appreciate that other medical devices having a diameter similar to, or slightly larger than, the diameter of a conventional wire guide may be inserted instead of, or in addition to, the wire guide (e.g., positioned over the wire guide). Non-limiting examples of such devices include small diameter catheters (e.g., about 0.035 to 0.07 inch [0.89 to 1.78 mm] diameter), obturators, and bougies.

FIGS. 5 and 6 illustrate longitudinal sectional views of airway management apparatus 10, also showing the catheter 90 and wire guide 96. In FIG. 5, the collar 46 is positioned over fingers 42. FIGS. 5A, and 5B are enlarged views showing detail of the fingers and collar, respectively. In the arrangement of FIG. 5, the fingers 42 are compressed in a radially inward direction against catheter 90, such that catheter 90 is locked into engagement with apparatus 10. In FIG. 6, the collar 46 is disposed proximal to the position shown in FIG. 5. In this arrangement, the compression exerted upon catheter 90 by the fingers is relaxed, whereupon the catheter may be removed from airway management apparatus 10.

As further shown in FIGS. 4-6, the proximal end 92 of catheter 90 is fully received in the chamber defined by fingers 42 and cylindrical base 43. In the preferred embodiment shown, catheter proximal end 92 engages an O-ring seal 49 positioned at the proximal end of port 40. O-ring seal 49 may be formed of conventional construction, such as nitrile rubber, a synthetic rubber copolymer. When catheter proximal end 92 is positioned against the O-ring seal, the catheter is pneumatically sealed in the chamber.

When the proximal end 92 of catheter 90 is initially inserted in the chamber, collar 46 is in a proximal position as shown in FIG. 6, and the distal ends of the fingers deflect in a radially outward direction. In one embodiment, each finger 42 may include a projection 48 (FIG. 5A) that extends inwardly into the chamber. The dimension between the opposing projections 48 of the flexible members is less than the outside diameter of the catheter 90. As a result, when the catheter proximal end 92 is inserted in the chamber, the distal ends of the fingers are urged in a radially outward direction as shown in FIG. 6.

Circumferentially arranged fingers 42 may have a conically shaped outer cam surface 45 along their distal length. The inside surface 52 (FIG. 5B) of ring-like collar 46 engages respective cam surfaces 45 when the collar is moved toward the distal end of the fingers as shown. As a result, projections 48 are forced in a radially inward direction in a gripping fashion against the outside surface of catheter 90.

As depicted in FIG. 5, collar 46 has been advanced to a fully distal position. Preferably, collar 46 includes a distal notched portion 47 that is sized and positioned to engage a cooperating retaining flange 54 of the respective fingers. When at the distal position, the inside surface of collar 46 fully engages the cam surfaces of the respective fingers, thereby forcing projections 48 against the outside surface of catheter 90. In this position, the catheter 90 is in a “locked” position relative to port 40. To unlock the catheter, collar 46 is slid in the proximal direction shown in FIG. 6, thereby disengaging the cam surface 45 of respective fingers 42. Fingers 42 may include a recessed surface or notch 41 adjacent can surface 45. This recessed surface limits the engagement of the collar with the fingers. As a result, the force necessary to move the collar to a proximal position and thereby “unlock” the port is minimized. Further discussion and explanation of this locking and rapid disconnect feature of port 40 is provided in the incorporated-by-reference '386 patent.

FIG. 7 is a side view of an alternative embodiment of an airway management apparatus 80. Airway management apparatus 80 has many features in common with apparatus 10, which features are provided with common numbers with the embodiment of FIGS. 1-6. In the embodiment of FIG. 7, airway management apparatus 80 includes an additional port 82. As illustrated, this port may be positioned opposite port 30 along the major axis of main body 12. Port 82 is shown with a Luer connection 84 for engagement with a mating connector of a medical or diagnostic device, e.g., a jet ventilator (not shown). Those skilled in the art will appreciate that other ports and port configurations, and connectors, may be provided in place of, or in addition to, port 82. Similarly, as stated above, other ports and port configurations may be provided in place of, or in addition to, port 30.

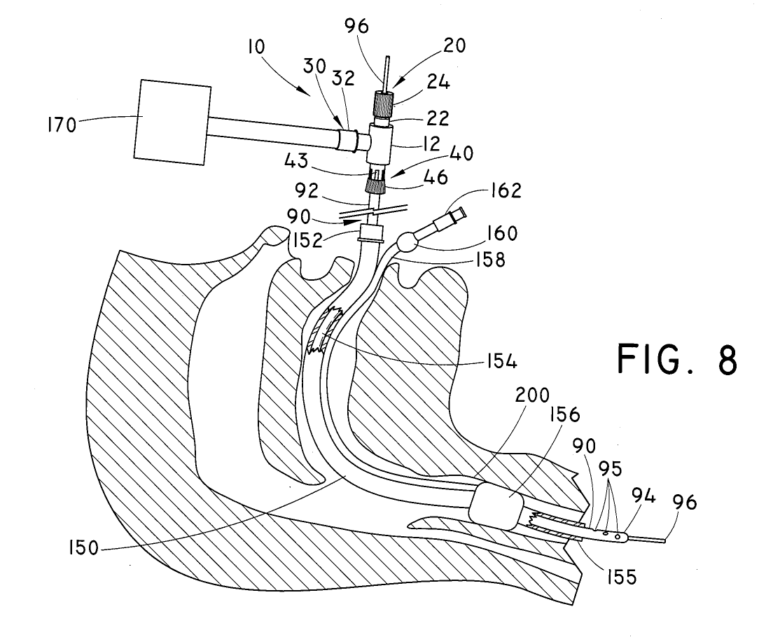

One example of use of the airway management apparatus 10 will now be described. In this example, airway exchange apparatus 10 is utilized to oxygenate a patient during replacement of an endotracheal tube (FIG. 8). Endotracheal tubes are well-known medical devices for providing ventilation to a patient. Endotracheal tube 150 includes a ventilator connector 152 at its proximal end. In this example, ventilator connector 152 is sized for connecting the endotracheal tube to a conventional ventilation apparatus 170 in well-known manner. A ventilation passageway 154 extends through the endotracheal tube for passage of a ventilating fluid through an open distal end 155 to ventilate the patient. Open distal end 155 is shown partially broken away to better illustrate the distal end of airway exchange catheter 90 that extends through the passageway of the endotracheal tube. Endotracheal tube 150 also includes an inflatable cuff 156 positioned in the vicinity of the distal end thereof to securely position and seal the distal end of the endotracheal tube in the trachea 200. Cuff 156 is inflated in conventional fashion with air supplied through an inflation tube 158 attached to the external surface of endotracheal tube 150. An inflatable pilot balloon 160 and a connector 162 may be provided at the proximal end of the inflation tube for use in inflating the cuff in well-known manner.

To prepare for endotracheal tube replacement, an airway exchange catheter 90 is engaged with port 40 of airway management apparatus 10. Proximal end 92 of the catheter is received in the chamber defined by fingers 42 and cylindrical base 43 as described hereinabove. Collar 46 is thereafter advanced to the distal position (FIG. 5) to securely engage catheter proximal end 92 with port 40 as described above.

The connection between the endotracheal tube 150 and ventilation apparatus 170 is interrupted by disconnecting the endotracheal tube 150 from the ventilation apparatus at ventilator connector 152. The ventilation apparatus 170 is thereafter connected to ventilator connector 32 at port 30 of the airway management apparatus 10, as shown in FIG. 8. The airway exchange catheter 90 is inserted through ventilator connector 152 and passageway 154 of the endotracheal tube 150. The distal end 94 of catheter 90 extends beyond endotracheal tube distal end 155 and into the trachea 200 to supply oxygen to the patient.

Wire guide 96 (or other medical apparatus as described above) is introduced into apparatus 10 via port 20. Typically, the wire guide is about twice as long as the airway exchange catheter, although these relative dimensions may be varied if desired. The wire guide is advanced through ports 20 and 40 along the major axis of airway management apparatus 10, and thereafter through catheter 90, such that the distal end of wire guide 96 extends beyond (i.e., distal to) the distal end of catheter 90 in the trachea, as shown in FIG. 8. The presence of the wire guide enables the operator to maintain access to the trachea, even if the catheter and endotracheal tube are removed, withdrawn, or otherwise dislodged from the trachea.

With a chronically placed endotracheal tube, it is not uncommon for the tissue of the patient's airway to become inflamed, and thereby encapsulate the endotracheal tube. When the endotracheal tube is removed, the inflamed tissue may hinder or prevent passage of air through the patient's airway, and may hinder access to the trachea. Thus, maintaining a wire guide (or a small diameter catheter, bougie, etc.), in the airway enables the medical professional to maintain access to the trachea, even after the endotracheal tube and/or airway exchange catheter have been withdrawn.

The endotracheal tube cuff is deflated, and the endotracheal tube is withdrawn from the trachea along the airway exchange catheter 90. Generally, when the endotracheal tube has been fully withdrawn from the trachea, the proximal end of the endotracheal tube substantially reaches port 40. At this stage, the patient is oxygenated by way of the ventilation fluid passing through one or more openings at the distal end of catheter 90. The presence of the side ports 95 insures a route for the oxygen to pass through the catheter 90 to the trachea even if the wire guide or other small diameter device blocks, or substantially blocks, passage of oxygen out the distal end opening of the catheter.

At this time, the airway management apparatus 10 may be removed from catheter 90 by disconnecting proximal end 92 of the catheter from airway management apparatus 10 at port 40 (FIGS. 5, 6), and withdrawing apparatus 10 over the wire guide. As stated above, and in the incorporated-by-reference '386 patent, this disconnect step is facilitated due to the rapid disconnect features provided at port 40. In the example described in the specification, the rapid disconnect features includes the fingers 42 and collar 46 shown in FIGS. 5, 6. Endotracheal tube 150 is removed from the trachea and airway of the patient over catheter 90, while the catheter and wire guide remain in place in the trachea of the patient. Following removal of the endotracheal tube, airway management apparatus 10 may be reinserted over the wire guide, and re-connected to catheter proximal end 92 at port 40 to re-establish a flow of oxygen to the patient.

An endotracheal tube is then arranged for re-insertion into the trachea. The endotracheal tube may be a new tube, or alternatively, may be the same endotracheal tube 150 that had been removed and cleaned. Catheter proximal end 92 is once again disconnected from port 40 of the airway management apparatus as described above, and apparatus 10 is withdrawn over the proximal end of wire guide 96. The endotracheal tube is then inserted over catheter proximal end 92 and wire guide 96, and advanced into the trachea. The catheter is reconnected to port 40 in the manner described above, thereby resuming the supply of oxygen to the patient, through the side ports 95 of catheter 90.

If difficulties are encountered during re-insertion of the endotracheal tube, the physician can remove the tube, re-establish oxygenation via catheter 90, and when deemed appropriate, repeat the endotracheal tube insertion process. The rapid disconnect features of port 40 as described above facilitate such removal and re-insertion. Once insertion of the endotracheal tube has been completed, airway exchange catheter 90 and wire guide 96 are withdrawn from the lumen of the endotracheal tube. Ventilator apparatus 170 is disconnected from the ventilator connector at port 30, and re-connected to ventilator connector 152 of the endotracheal tube.

Those skilled in the art will appreciate that not all steps described above need be performed in the exact order described in this example in all instances. Further, it may not be necessary to carry out each step described in the example in every instance, and those skilled in the art are capable of determining whether any modification of the described process is appropriate. However, it is believed that optimal results will be obtained in most instances when the tubular replacement process is carried out in the manner described herein.

Unlike the rapid disconnect feature described in the '386 patent, the airway management apparatus 10 of the present invention is capable of receiving wire guide 96, while at the same time being operably connected to the ventilation apparatus as described in the example, or to an alternative ventilation connector, such as a connector suitable for use with a jet-type ventilator. Thus, access to the trachea is maintained throughout the endotracheal tube replacement process.

It is therefore intended that the foregoing detailed description be regarded as illustrative rather than limiting, and that it be understood that it is the following claims, including all equivalents, that are intended to define the spirit and scope of this invention.

What is claimed is:

1. An airway management apparatus for engagement with a catheter for oxygenation of a patient, comprising:
a generally hollow main body having a plurality of ports open to an interior space thereof, said main body having a major axis, a first port extending in a proximal direction from said main body along said major axis, a second port angularly offset from said major axis, and a third port extending in a distal direction from said main body wherein said third port is at least substantially in-line with said first port along said major axis, said third port comprising a connector for engagement with a proximal end of said catheter, said connector comprising a plurality of radially compressible members extending in a distal direction, said compressible members circumferentially aligned to define a chamber for receiving said catheter proximal end, said connector further comprising an axially movable member positioned for selectively compressing a distal end portion of said compressible members around said catheter proximal end, and for releasing said compressible members from around said catheter proximal end.

2. The airway management apparatus of claim 1, wherein said first port comprises a valve member sized and configured for passage of said catheter therethrough.

3. The airway management apparatus of claim 2, wherein said second port is configured for engagement with a ventilation apparatus.

4. The airway management apparatus of claim 3, wherein said compressible members comprise respective fingers extending in said distal direction to define said chamber, and wherein said axially movable member comprises a collar for selectively compressing a distal end portion of said fingers around said catheter proximal end, and for releasing said fingers from around said catheter proximal end.

5. The airway management apparatus of claim 4, wherein said fingers include respective projections radially movable upon a distal movement of said collar for grippingly engaging said catheter proximal end.

6. The airway management apparatus of claim 4, further comprising a fourth port angularly offset from said major axis.

7. An airway management system for oxygenating a patient, comprising:
a catheter having a proximal end, a distal end, and a passageway extending therethrough, said catheter having an outer surface dimensioned to be received in a passageway through an endotracheal tube; and
an apparatus comprising a generally hollow main body and a plurality of ports open to an interior space of said main body, a first port disposed at a proximal portion of said apparatus, a second port, and a third port disposed at a distal portion of said apparatus, said third port positioned substantially in-line with said first port along an axis of said apparatus, said third port comprising a connector sized and configured for releasably engaging said proximal end of said catheter, said connector comprising a radially compressible member extending in a distal direction for receiving said catheter proximal end, said connector further comprising an axially movable member positioned for selectively compressing said compressible member around said catheter proximal end, and for releasing said compressible member from around said catheter proximal end.

8. The airway management system of claim 7, wherein said compressible member comprises a plurality of radially compressible fingers extending in said distal direction, said fingers circumferentially aligned to define a chamber for receiving said catheter proximal end, said axially movable member comprising a collar for selectively compressing a distal end portion of said fingers around said catheter proximal end, and for releasing said fingers from around said catheter proximal end.

9. The airway management system of claim 8, further comprising a guide device configured to extend through said first port and said third port along said axis, and through said catheter when said catheter is engaged with said third port.

10. The airway management system of claim 9, wherein said guide device comprises a wire guide, and wherein said catheter comprises one or more side ports disposed along a distal portion thereof, said side ports in communication with said catheter passageway.

11. The airway management system of claim 10, wherein said first port comprises a valve member, and wherein said catheter is extendable through said valve member.

12. The airway management system of claim 8, further comprising a ventilation apparatus, and wherein said second port is configured for engagement with said ventilation apparatus.

13. The airway management system of claim 12, further comprising an endotracheal tube sized to receive said catheter.

14. The airway management system of claim 8, wherein said apparatus comprises a fourth port angularly offset from said axis.

15. A method for oxygenating a patient during removal of an endotracheal tube, wherein a proximal end of the endotracheal tube is engaged with a ventilation apparatus and a distal end extends into the trachea of the patient, comprising:
positioning a multi-port airway apparatus for engagement with a catheter, said multi-port airway apparatus comprising a main body having a proximal first port, a second port, and a distal third port, said third port at least substantially in-line with said first port and comprising a connector member;
engaging a proximal end of said catheter with said connector member of said third port;
disengaging the proximal end of the endotracheal tube end from the ventilation apparatus, and engaging the ventilation apparatus with said second port;
passing a distal end of said catheter through a passageway of the endotracheal tube, such that the catheter distal end extends beyond the distal end of the endotracheal tube; and
passing a guide device through the first and third ports of the multi-port airway apparatus, and through a lumen of the catheter, such that a distal end of the guide device extends into the trachea.

16. The method of claim 15, comprising:
at least partially withdrawing the endotracheal tube from the trachea over the catheter and guide device;
disengaging the proximal end of the catheter from the connector member of the third port, and withdrawing the airway apparatus over the guide device; and
removing the endotracheal tube over the guide device.

17. The method of claim 16, comprising:
passing said airway apparatus over said guide device; and
re-engaging said catheter proximal end with said connector member.

18. The method of claim 17, comprising:
disengaging the proximal end of the catheter from the connector member, and withdrawing the airway apparatus over the guide device;
inserting an endotracheal tube over the catheter proximal end, and advancing the distal end of the endotracheal tube into the trachea; and
advancing the airway apparatus over the guide device, and re-engaging said catheter proximal end with said connector member.

19. The method of claim 18, comprising:
withdrawing said airway apparatus, catheter, and guide device;
disengaging the ventilation apparatus from said second port; and
engaging the proximal end of the endotracheal tube end with the ventilation apparatus.

20. The method of claim 15, wherein said catheter distal end comprises a plurality of side ports, and said guide device comprises a wire guide.
